SEQUENCE LISTING (2 sequences)

SEQ ID NO: 1 - protein, 39 aa, Artificial Sequence
  Fully synthetic peptide
HGEGTFTSDLSKQMEEEAVRLFIEWLKNGGPSSGAPPPS

SEQ ID NO: 2 - protein, 39 aa, Artificial Sequence; identical to SEQ ID NO: 1
  Fully synthetic peptide

(19) United States
(12) Patent Application Publication
(10) Pub. No.: US 2013/0289241 A1
(43) Pub. Date: Oct. 31, 2013

(54) METHOD FOR PREPARING EXENATIDE

(71) Applicants: Juncai BAI, Shanghai (CN); Guoqing ZHANG, Shanghai (CN); Ruoping ZHANG, Shanghai (CN)

(72) Inventors: Juncai BAI, Shanghai (CN); Guoqing ZHANG, Shanghai (CN); Ruoping ZHANG, Shanghai (CN)

(73) Assignee: SHANGHAI AMBIOPHARM, INC., Shanghai (CN)

(21) Appl. No.: 13/457,482

(22) Filed: Apr. 26, 2012

(51) Int. Cl. C07K 1/04

(52) U.S. Cl. 530/334

(57) ABSTRACT

A method for preparing exenatide by solid-phase synthesis, including: 1) mixing an Fmoc-Rink amide AM resin with a deprotecting agent to obtain a Rink amide AM resin; 2) condensing an Fmoc-Ser(tBu)-OH with the Rink amide AM resin to obtain an Fmoc-Ser(tBu)-Rink amide AM resin; 3) repeating the Fmoc deprotection and the condensation between an amino acid and a polypeptide on the resin, and condensing an amino acid with a polypeptide on the resin from the C-terminal to the N-terminal, to form a polypeptide resin; and 4) separating the polypeptide and the resin on the polypeptide resin.

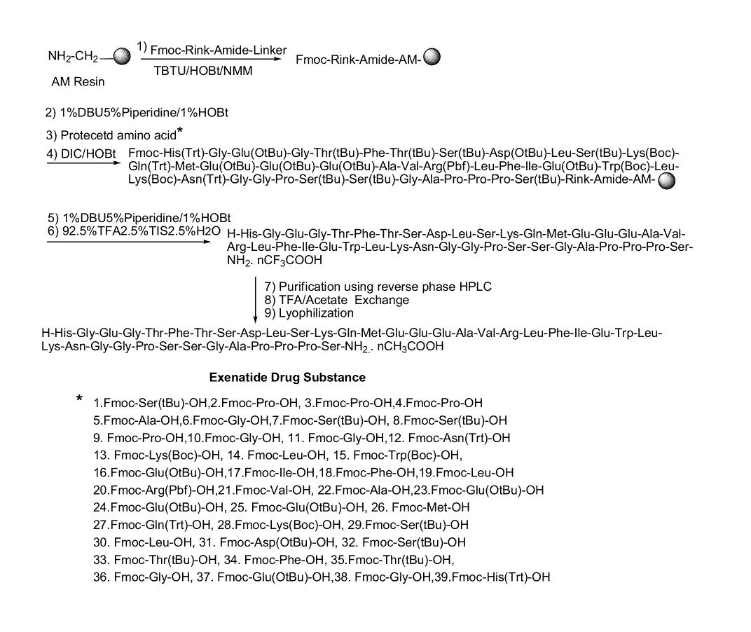

### BACKGROUND OF THE INVENTION

1. Field of the Invention

The invention relates to the field of solid-phase polypeptide synthesis, and more particularly to a solid-phase synthesis method of exenatide.

2. Description of the Related Art

Exenatide is a 39-amino-acid polypeptide, and serves as the first incretin mimetics. The incretin mimetics is a novel approach for the treatment of type II diabetes, and can imitate the antidiabetic reactions or the reactions of reducing glucose concentration of natural gastrointestinal hormones in human body. These reactions include stimulating the production of insulin in the body when blood glucose rises, inhibiting the secretion of glucagons at the end of the meal, decelerating the speed of drawing nutrients from the blood, and lowering the food intakes. Exenatide is a completely new therapeutic drug for type II diabetes. Exenatide is taken twice every day by hypodermic injection, and used for the type II diabetes patients with blood glucose uncontrolled by metformin, sulfonylurea, or the combination of metformin and sulfonylurea.

At present, the exenatide synthesis method includes a solid-phase synthesis method and a combined solid-liquid phase synthesis method. Three different peptide intermediate fragments are synthesized by a solid-phase chemical method, another amino acid substance is added to one of the fragments by a solution-phase chemical method, and then these fragments are coupled together in the solution phase.

The liquid-phase synthesis steps of exenatide are complex, the controlling items produced by GMP are more, the stability is poor, and the produced byproducts are complicated. The foreign impurities are removed through times of washing or other purification methods, thus the cost is high.

### SUMMARY OF THE INVENTION

In view of the above-described problems, it is one objective of the invention to provide a method for preparing exenatide by solid-phase synthesis.

To achieve the above objective, in accordance with one embodiment of the invention, there is provided a method for preparing exenatide represented by SEQ. ID NO. 1 by solid-phase synthesis. The method comprises the following steps:
(1) mixing an Fmoc-Rink amide AM resin with a deprotecting agent to obtain a Rink amide AM resin;
(2) condensing an Fmoc-Ser(tBu)-OH with the Rink amide AM resin to obtain an Fmoc-Ser(tBu)-Rink amide AM resin;
(3) repeating the Fmoc deprotection in step (1) and the condensation between an amino acid and a polypeptide on the resin in step (2) according to a solid-phase synthesis method, and condensing an amino acid with a polypeptide on the resin from the C-terminal to the N-terminal according to the sequence from Ser to His, to form a polypeptide resin represented by SEQ ID NO. 1; and
(4) separating the polypeptide on the polypeptide resin represented by SEQ. ID NO. 1 from the resin, to obtain exenatide represented by SEQ. ID NO. 2.

SEQ. ID. NO. 1
Fmoc-His(Trt)-Gly-Glu(OtBu)-Gly-Thr(tBu)-Phe-
Thr(tBu)-Ser(tBu)-Asp(OtBu)-Leu-Ser(tBu)-
Lys(Boc)-Gln(Trt)-Met-Glu(OtBu)-Glu(OtBu)-
Glu(OtBu)-Ala-Val-Arg(Pbf-Leu-Phe-Ile-
Glu(OtBu)-Trp(Boc)-Leu-Lys(Boc)-Asn(Trt)-Gly-
Gly-Pro-Ser(tBu)-Ser(tBu)-Gly-Ala-Pro-Pro-
Pro-Ser(tBu)-Rink amide AM resin
SEQ. ID. NO. 2
H-His-Gly-Glu-Gly-Thr-Phe-Thr-Ser-Asp-Leu-Ser-
Lys-Gln-Met-Glu-Glu-Glu-Ala-Val-Arg-Leu-Phe-
Ile-Glu-Trp-Leu-Lys-Asn-Gly-Gly-Pro-Ser-Ser-
Gly-Ala-Pro-Pro-Pro-Ser-NH₂.nCH₃COOH

In a class of this embodiment, after the step of deprotection of Fmoc group and the step of condensation between the amino acid and the Rink amide AM resin or the polypeptide Rink amide AM resin, the Rink amide AM resin or the polypeptide Rink amide AM resin is washed with an N,N-dimethylformamide (DMF) solution with concentration of 50-100 v/v %.

In a class of this embodiment, the deprotecting agent, on the basis of its total volume, comprises 3-20% of piperidine, 0.5-10% of Bicyclic amidine (DBU), and 0.5-10% of 1-hydroxybenzotriazole (HOBt).

In a class of this embodiment, the condensing is carried out in the presence of a condensing agent. The condensing agent is a mixture of N,N′-Diisopropylcarbodiimide (DIC) and one or more of 0-(benzotriazole-1-yl)-N,N,N′,N′-tetramethyl uronium tetrafluoroborate (TBTU), diisopropylethylamine (DIPEA), and 1-hydroxybenzotriazole (HOBt).

In a class of this embodiment, the condensing agent N,N′-Diisopropylcarbodiimide (DIC) is added twice, and after the first addition, allow to react for 20-60 min, then perform the second addition, and allow to react for 60-180 min.

In a class of this embodiment, the condensing is carried out under the monitoring of a Ninhydrin test method.

In a class of this embodiment, the amino remaining after the condensation reaction is acetylated with acetic anhydride.

In a class of this embodiment, step (4) is carried out in the presence of a cutting agent comprising trifluoroacetic acid (TFA), triisopropylsilane (TIS), thioanisole, and water.

Advantages of the invention are summarized below:
1. The synthesis process of exenatide prepared by a solid-phase synthesis method provided by the invention has simple steps, convenient operation, and controllable cost; and
2. The purity of the product obtained by the solid-phase synthesis method is high (>98%).

### BRIEF DESCRIPTION OF THE DRAWINGS

The invention is described hereinbelow with reference to accompanying drawings, in which the sole FIGURE is a flow chart for preparing exenatide by solid-phase synthesis.

### DETAILED DESCRIPTION OF THE EMBODIMENTS

After the intensive and extensive studies, the inventor discovers a process of preparing exenatide by solid-phase synthesis, and the condensing agent N,N′-Diisopropylcarbodiimide (DIC) is added twice to ensure the complete reaction. The amino remaining after the condensation reaction is capped with acetic anhydride, so as to reduce side reactions and foreign impurities. N,N-dimethylformamide (DMF) is used for washing after deprotection and condensation of each time, so as to achieve a good solubility and swellability for all the reagents used in the condensation process.

The abbreviations or the implications of English full names used in the preparation method of Exenatide are listed in the table below.

| | |
|---|---|
| Fmoc | 9-fluorenylmethoxycarbonyl |
| DMF | N,N-dimethylformamide |
| DBU | 1,8-diazabicyclo(5.4.0)undec-7-ene |
| HOBt | 1-hydroxy benzotriazole |
| DIC | N,N′-diisopropylcarbodiimide |
| TBTU | O-(benzotriazole-1-yl)-N,N,N,N- |
| | 4-methyl-uronium tetrafluoroborate |
| DIPEA | Diisopropyl ethylamine |
| NMM | N-methyl morpholine |
| Ac₂O | Acetic anhydride |
| TFA | Trifluoroacetic acid |
| TIS | Triisopropyl silane |
| MTBE | Methyl tert-butyl ether |
| Boc | Butoxycarbonyl |
| tBu | Tert-butyl, —C(CH₃)₃ |
| OtBu | —O—C(CH₃)₃ |

In embodiments of the invention, “solid phase synthesis” or “solid phase peptide phase” is well-known to one of ordinary skill in the art, comprising but not limited to the following steps: a) covalently binding a first amino acid whose amino-group is blocked to a solid phase carrier; b) in the presence of a de-protective agent, removing the protecting group of the amino-group; c) activating the carboxyl of a second amino acid whose amino-group is blocked and contacting the second amino acid with the first amino acid bound to the solid phase carrier so that a dipeptide whose amino-group is blocked is obtained; d) repeating the peptide bond formation steps and thus the peptide chain is extended from C-terminal to N-terminal; and e) removing the protecting group of the amino-group and separating the peptide chain from the solid phase carrier with a cleavage agent to yield a peptide.

In embodiments of the invention, the “protection eliminating agent” or “deprotecting agent” can be used interchangeably, and is a chemical reagent for removing amino-protecting agents connected on amino acids, and the amino-protecting agent can be well-known in the field, such as, but not limited to Fmoc and Boc; preferably, the protection eliminating agent is calculated in total volume and is a DMF solution containing 3-20 v/v % of piperidine, 0.5-10 v/v % of Bicyclic amidine (DBU), and 0.5-10 w/v % of 1-hydroxybenzotriazole (HOBt).

In embodiments of the invention, the “condensing agent”, “activating agent”, or “condensation activating agent” can be used interchangeably, which are a chemical reagent for allowing an amino group from one amino acid to be condensed with a carboxyl group from another amino acid to form peptide bonds, and well-known in the field, such as, but not limited to DIC, HATU, TBTU, and DIPEA.

In embodiments of the invention, the cleavage agent is a chemical agent which can separate a peptide bound to a resin from the resin. The cleavage agent is well-known to those of ordinary skill in the art and includes but is not limited to a weak acid solution comprising TFA and HCl solution.

In embodiments of the invention, the “Rink Amide Linker” is a connecting arm used in the polypeptide synthesis, its structure is shown in formula III, its molecular formula is C₃₂H₂₉NO₇, its molecular weight is 539.58, and its CAS number is 145069-56-3.

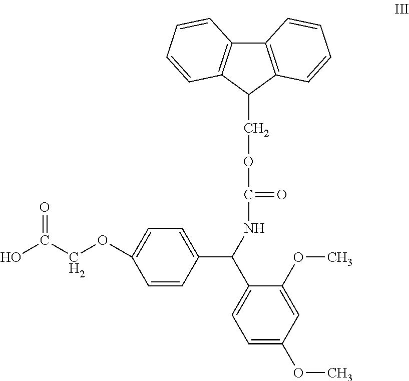 [SMILES: COc1ccc(C(NC(=O)OCC2c3ccccc3-c3ccccc32)c2ccc(OCC(=O)O)cc2)c(OC)c1]

In an example of the preparation method of exenatide, the method for preparing exenatide by solid-phase polypeptide synthesis comprises the following steps:
Step 1, mixing an AM polystyrene resin, Fmoc-Rink-Amide-Linker, TBTU, and HOBt with NMM to obtain an Fmoc-Rink amide AM resin with substitution degree of 0.4-1.6 mmol/g;
Step 2, mixing a protection eliminating agent with the Fmoc-Rink amide AM resin to obtain a Rink amide AM resin by removing the Fmoc group;
Step 3, condensing Fmoc-Ser(tBu)-OH with the Rink amide AM resin to obtain an Fmoc-Ser(tBu)-Rink amide AM resin;
Step 4, removing the Fmoc group using the protection eliminating agent;
Step 5, repeating the steps of formation of peptide bonds, so as to enable the peptide chain to grow from the C terminal to the N terminal until to obtain Fmoc-His(Trt)-Gly-Glu(OtBu)-Gly-Thr(tBu)-Phe-Thr(tBu)-Ser(tBu)-Asp(OtBu)-Leu-Ser(tBu)-Lys(Boc)-Gln(Trt)-Met-Glu(OtBu)-Glu(OtBu)-Glu(OtBu)-Ala-Val-Arg(Pbf)-Leu-Phe-Ile-Glu(OtBu)-Trp(Boc)-Leu-Lys(Boc)-Asn(Trt)-Gly-Gly-Pro-Ser(tBu)-Ser(tBu)-Gly-Ala-Pro-Pro-Pro-Ser(tBu)-Rink amide AM (OtBu/tBu is a protective group, and finally removed);
Step 6, removing the Fmoc group using the protection eliminating agent; and
Step 7, separating the polypeptide (represented by SEQ. ID NO. 2) on the polypeptide resin from the resin to obtain exenatide in the presence of a cutting agent; the cutting agent, comprising TFA, TIS, thioanisole, and water.

Preferably, the amino remaining after reaction on the resin in step 1 is capped with acetic anhydride/pyridine/DMF.

Preferably, the deprotection is continuously carried out twice.

Preferably, in the reaction of formation of the peptide bonds in step 3 and/or step 5, Fmoc-amino acid or polypeptide (0.5-3 Fmoc-Rink amide AM resin equivalent) and DMF solution comprising HOBt (0.5-3 Fmoc-Rink amide AM resin equivalent) are mixed with DIC (1-3 Fmoc-Rink amide AM resin equivalent) to react for 20-60 min, and then DIC (1-3 Fmoc-Rink amide AM resin equivalent) is added again to react for 60-180 min. The Ninhydrin test method is used for monitoring the condensation reaction. If the reaction is incomplete, Fmoc-amino acid or polypeptide (0.5-3 Fmoc-Rink amide AM resin equivalent), TBTU (1-3 Fmoc-Rink amide AM resin equivalent), HOBt (1-3 Fmoc-Rink amide AM resin equivalent), and DMF solution comprising DIPEA (1-3 Fmoc-Rink amide AM resin equivalent) are added to ensure the complete reaction. If the recondensation is incomplete, the amino remaining after reaction is acetylated with an acylation reagent, and the acylation reagent is selected from acetic anhydride, benzoyl chloride or 2,6-Dichlorobenzoyl chloride, such as but not limited to 15-35% Ac₂O/Pyridine/DMF (V/V/V).

The ninhydrin colorimetric method (Kaiser) and Chloranil and Kaiser test method is recited in the literatures below: VIRENDER K. SARIN, et al. “Quantitative Monitoring of Solid-Phase Peptide Synthesis by the Ninhydrin Reaction” ANALYTICAL BIOCHEMISTRY 117, 147-157 (1981); E. KAISER, et al. “Color Test for Detection of Free Terminal Amino Groups in the Solid-Phase Synthesis of Peptides” SHORT COMMUNICATIONS 595-598 (Received Oct. 28, 1969); and THORKILD CHRISTENSEN “A Qualitative Test for Monitoring Coupling Completeness in Solid Phase Peptide Synthesis Using Chloranil” Acta Chemica Scandinavica B 33 (1979) 763-766.

Preferably, the exenatide obtained in step 7 is precipitated, that is, the polypeptide represented by SEQ. ID NO. 2 and obtained in step 7 is mixed with MTBE or ether to form a polypeptide precipitate. The polypeptide precipitate is filtered to obtain crude exenatide, and then the crude exenatide is purified by preparative chromatography.

The above mentioned technical features can be combined freely upon implementation.

For further illustrating the invention, experiments detailing a method for preparing exenatide using a solid-phase synthesis method are described below. It should be noted that the following examples are intended to describe and not to limit the invention.

Unless otherwise specified, the experiments in Examples are carried out at normal conditions or in accordance with the conditions recommended by the manufacturer, and all percentage, ratio, or proportion is calculated by weight.

The volume percentage of weight of the invention is well-known to those of ordinary skill in the art, e.g., the weight of solute dissolved in 100 mL of solution.

Unless otherwise specified, the meaning of scientific terms in the invention is the same as that known to those of ordinary skill in the art. Methods or materials similar to or equal to those of the invention are practical.

The method for detecting the purity of exenatide is as follows:

Chromatographic column: Vydac 218TP C18 5u SG. 658

Column temperature: 45° C.

Mobile phase: A: 0.1%TFA/water; B: 0.1%TFA/acetonitrile

Flow rate: 1.0 mL/min

Gradient elution: 27-47% of mobile phase B in 30 min

The purification method of exenatide is as follows: the crude peptide is purified with a reverse phase HPLC chromatographic column (Hanbon ODS-2 50×250 mm, C18), and mobile phase A: 50 mM ammonium acetate aqueous; mobile phase B: acetonitrile with 20 v/v % mobile phase A (i.e., ACN with 20% A); gradient elution: 28-48% mobile phase B in 100 min, the flow rate of the mobile phase is 80 mL/min, the solution with purity higher than 80% is collected and purified with the same chromatographic column, and mobile phase C: Water with 0.085% phosphoric acid; mobile phase D: acetonitrile with 20 v/v % mobile phase C (i.e., ACN with 20% C); gradient elution: 28-48% mobile phase D in 80 min, and the flow rate of the mobile phase is 80 mL/min.

### EXAMPLE 1

Loading of Fmoc-Rink Amide Linker

24.0 g AM polystyrene resin (substitution degree: 0.6-0.9 mmole/g) reacts with Fmoc-Rink-Amide-Linker (equivalent: 1.0), TBTU (equivalent: 1.425), HOBT (equivalent: 1.5), and NMM (equivalent: 3) for 3 hours with stirring, the amino remaining after reaction on the resin is capped with Ac2O/Pyridine/DMF (v/v/v). 34 g resin is finally obtained, with a substitution degree of 0.58 mmole/g.

Deprotection

The deprotection is continuously carried out twice for 10 min and 20 min with 8% piperidine/1.5 DBU/5% HOBt/DMF (v/v/w/v). DMF and methanol are used for washing; the removal of the Fmoc group is monitored and evaluated by Kaiser test after the thorough draining.

Condensation of Amino Acids

Fmoc-AA-OH/HOBt (equivalent 1.0/equivalent 1.0) and DMF solution (1.0 Fmoc-Rink amide AM resin equivalent) are added to a reactor, and then DIC (1.5 Fmoc-Rink amide AM resin equivalent) is added. 30 min later with stirring, DIC (1.5 Fmoc-Rink amide AM resin equivalent) is added again, and then the reaction is carried out for at least one hour with stirring.

Recondensation and Acetylation

After an hour of reaction, the Ninhydrin test method is used for monitoring, if the reaction is incomplete, the Fmoc-AA-OH (equivalent: 1.0)/TBTU (equivalent: 1.0)/HOBT (equivalent: 1.0)/DIPEA (equivalent: 1.0)/DMF solution is added for reaction for at least 3 hours, and the Ninhydrin test method is used for monitoring.

If the reaction is still incomplete after recondensation, the amino remaining after reaction is acetylated in 25% Ac2O/18% Pyridine/57% DMF (v/v/v) with acetic anhydride. The DMF/MeOH/MTBE is used for washing the resin after the completion of total condensation reaction and the removal of the Fmoc group, and then the washed resin is dried and weighed.

Cutting

The cooled TFA/TIS/thioanisole/water (80-95% v/1-10% v/1-10% v/1-10% v) is used as cutting fluids, and the reaction is carried out 2-3 hours after the temperature rises to 25° C.±5° C. with stirring.

The condensed filtrate is poured into the cooled methyl tertiary butyl ether (MTBE) for sedimentation; the crystallization is carried out for 0.5-1.5 hours after cooling and standing; the filter cake is obtained by filtering or centrifuging, and then thoroughly washed for three times with frozen methyl tertiary butyl ether (MTBE); the crude polypeptide is transferred to a drier and dried for at least 12 hours under vacuum.

The purity is 40%.

Purification

The purification is carried out by preparative chromatography, the product with purity of 98.4% is obtained, and the purification yield is 12.4%.

### EXAMPLE 2

Loading of Fmoc-Rink Amide Linker

24.0 g AM polystyrene resin (substitution degree: 0.8-1.0 mmole/g) reacts with

Fmoc-Rink-Amide-Linker (equivalent: 1.5), TBTU (equivalent: 1.425), HOBT (equivalent: 1.5), and NMM (equivalent: 3) for 3 hours with stirring, the amino remaining after reaction on the resin is capped with Ac2O/Pyridine/DMF, then 34 g resin is finally obtained, with a substitution degree of 0.60 mmole/g.

Deprotection

The deprotection is continuously carried out twice for 10 min and 20 min with 6% piperidine/1.3 DBU/2% HOBt/DMF (v/v/w/v); the DMF and methanol are used for washing; the removal of the Fmoc group is monitored and evaluated by Kaiser test after the thorough draining.

Condensation of Amino Acids

Fmoc-AA-OH/HOBt (equivalent 1.5/equivalent 1.5) and DMF solution (1.5 Fmoc-Rink amide AM resin equivalent n) are added to a reactor, and then DIC (2.0 Fmoc-Rink amide AM resin equivalent) is added. 45 min later with stirring, DIC (2.0 Fmoc-Rink amide AM resin equivalent) is added again, and then the reaction is carried out for at least one hour with stirring.

Recondensation and Acetylation

After an hour of reaction, the Ninhydrin test method is used for monitoring, if the reaction is incomplete, Fmoc-AA-OH (equivalent: 1.5)/TBTU (equivalent: 1.5)/HOBT (equivalent: 1.5)/DIPEA (equivalent: 1.5)/DMF solution is added for reaction for at least 3 hours, and the Ninhydrin test method is used for monitoring.

If the reaction is still incomplete after recondensation, the amino remaining after reaction is acetylated with acetic anhydride. The DMF/MeOH/MTBE is used for washing the resin after the completion of total condensation reaction and the removal of the Fmoc group, and then the washed resin is dried and weighed.

Cutting

The cooled TFA/TIS/thioanisole/water (80-95% v/1-10% v/1-10% v/1-10% v) is used as cutting fluids, and the reaction is carried out 2-3 hours after the temperature rises to 25° C.±5° C. with stirring.

The condensed filtrate is poured into the cooled methyl tertiary butyl ether (MTBE) for sedimentation; the crystallization is carried out for 0.5-1.5 hours after cooling and standing; the filter cake is obtained by filtering or centrifuging, and then thoroughly washed for three times with frozen methyl tertiary butyl ether (MTBE); the crude polypeptide is transferred to a drier and dried for at least 12 hours under vacuum.

The purity is 43%.

Purification

The purification is carried out by preparative chromatography, the product with purity of 98.6% is obtained, and the purification yield is 12.8%.

### EXAMPLE 3

Loading of Fmoc-Rink Amide Linker

24.0 g AM polystyrene resin (substitution degree: 0.9-1.2 mmole/g) reacts with Fmoc-Rink-Amide-Linker (equivalent: 2.0), TBTU (equivalent: 1.425), HOBT (equivalent: 1.5) and NMM (equivalent: 3) for 3 hours with stirring, the amino remaining after reaction on the resin is capped with Ac2O/Pyridine/DMF, then 34 g resin is finally obtained, with a substitution degree of 0.57 mmole/g.

Deprotection

The deprotection is continuously carried out twice for 10 min and 20 min with 3% piperidine/1.0 DBU/6% HOBt/DMF (v/v/w/v); the DMF and methanol are used for washing; the removal of the Fmoc group is monitored and evaluated by Kaiser test after the thorough draining.

Condensation of Amino Acids

Fmoc-AA-OH/HOBt (equivalent 2.0/equivalent 2.0) and DMF solution (2.0 Fmoc-Rink amide AM resin equivalent) are added in a reactor, and then DIC (1.5 Fmoc-Rink amide AM resin equivalent) is added. 45 min later with stirring, DIC (1.5 Fmoc-Rink amide AM resin equivalent) is added again, and then the reaction is carried out for at least one hour with stirring.

Recondensation and Acetylation

After an hour of reaction, the Ninhydrin test method is used for monitoring, if the reaction is incomplete, the Fmoc-AA-OH (equivalent: 2.0)/TBTU (equivalent: 2.0)/HOBT (equivalent: 2.0)/DIPEA (equivalent: 2.0)/DMF solution is added for reaction for at least 3 hours, and the Ninhydrin test method is used for monitoring.

If the reaction is still incomplete after recondensation, the amino remaining after reaction is acetylated with acetic anhydride. The DMF/MeOH/MTBE is used for washing the resin after the completion of total condensation reaction and the removal of the Fmoc group, and then the washed resin is dried and weighed.

Cutting

The cooled TFA/TIS/thioanisole/water (80-95% v/1-10% v/1-10% v/1-10% v) is used as cutting fluids, and the reaction is carried out 2-3 hours after the temperature rises to 25° C.±5° C. again with stirring.

The condensed filtrate is poured into the cooled methyl tertiary butyl ether (MTBE) for sedimentation; the crystallization is carried out for 0.5-1.5 hours after cooling and standing; the filter cake is obtained by filtering or centrifuging, and then thoroughly washed for three times with frozen methyl tertiary butyl ether (MTBE); the crude polypeptide is transferred to a drier and dried for at least 12 hours under vacuum.

The purity is 45%.

Purification

The purification is carried out by preparative chromatography, the product with purity of 98.0% is obtained, and the purification yield is 12.2%.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

1. A method for preparing exenatide, the method comprising the following steps:
1) mixing an Fmoc-Rink amide AM resin with a deprotecting agent to obtain a Rink amide AM resin, said deprotecting agent comprising by volume: between 3 and 20% of piperidine, between 0.5 and 10% of 1,8-diazabicyclo(5.4.0)undec-7-ene, and between 0.5 and 10% of 1-hydroxybenzotriazole;
2) mixing an Fmoc-Ser(tBu)-OH with the Rink amide AM resin to obtain an Fmoc-Ser(tBu)-Rink amide AM resin through a condensation reaction, and blocking unreacted amino groups on said Rink amide AM resin with a reagent selected from the group consisting of acetic anhydride, benzoyl chloride, and 2,6-dichlorobenzoyl chloride;
3) repeating step (1) and step (2) according to a solid-phase synthesis method, and condensing amino acids successively to form a polypeptide resin comprising a polypeptide bound thereto, said polypeptide resin being represented by SEQ. ID NO. 1; and
4) separating the polypeptide from the polypeptide resin represented by SEQ. ID NO. 1, to obtain exenatide represented by SEQ. ID NO. 2.

2. The method of claim 1, wherein the method further comprises washing the Rink amide AM resin, the Fmoc-Ser(tBu)-Rink amide AM resin, and a polypeptide bound Rink amide AM resin obtained after each repetition of steps 1) and 2) with an N,N-dimethylformamide aqueous solution having a concentration of 50-100 v/v %.

3. (canceled)

4. The method of claim 1, wherein the condensation reaction is carried out in the presence of a condensing agent, and the condensing agent is a mixture of N,N′-diisopropylcarbodiimide and one or more of o-(benzotriazole-1-yl)-N,N,N′,N′-tetramethyl uronium tetrafluoroborate, diisopropylethylamine, and 1-hydroxybenzotriazole).

5. The method of claim 4, wherein N,N′-diisopropylcarbodiimide is added twice, after the first addition, the condensation reaction is allowed to proceed for 20-60 min, and after the second addition, the condensation reaction is allowed to proceed for 60-180 min.

6. The method of claim 1, wherein completion of the condensation reaction is monitored by a Ninhydrin test.

7. The method of claim 1, wherein the reagent acetic anhydride.

8. The method of claim 1, wherein step (4) is carried out in the presence of a cutting agent comprising trifluoroacetic acid, triisopropylsilane, thioanisole, and water.

9. A method for preparing exenatide, the method comprising:
1) mixing an Fmoc-Rink amide AM resin with a deprotecting agent to obtain a Rink amide AM resin, said deprotecting agent comprising between 3 and 20% v/v of piperidine, between 0.5 and 10% v/v of 1,8-diazabicyclo(5.4.0)undec-7-ene, and between 0.5 and 10% v/v of 1-hydroxybenzotriazole;
2) mixing 1-hydroxybenzotriazole and Fmoc-Ser(tBu)-OH with said Rink amide AM resin, adding N,N′-diisopropylcarbodiimide in a first amount of between 1 and 3 mole equivalents relative to said Fmoc-Rink amide AM resin and allowing said Fmoc-Ser(tBu)-OH and said Rink amide AM resin to react for between 20 and 60 min, then adding N,N′-diisopropylcarbodiimide in a second amount of between 1 and 3 mole equivalents relative to said Fmoc-Rink amide AM resin, and allowing said Fmoc-Ser(tBu)-OH and said Rink amide AM resin to react for between 60 and 180 min to obtain an Fmoc-Ser(tBu)-Rink amide AM resin;
3) blocking unreacted amino groups on said Rink amide AM resin with a reagent selected from the group consisting of acetic anhydride, benzoyl chloride, and 2,6-dichlorobenzoyl chloride;
4) repeating steps 1), 2), and 3), and condensing amino acids successively to form a polypeptide resin comprising a polypeptide bound thereto, said polypeptide resin being represented by SEQ. ID NO. 1; and
5) separating the polypeptide from the polypeptide resin represented by SEQ. ID NO. 1 to obtain exenatide represented by SEQ. ID NO. 2.

10. The method of claim 9, wherein the method further comprises washing the Rink amide AM resin, the Fmoc-Ser(tBu)-Rink amide AM resin, and a polypeptide bound Rink amide AM resin obtained after each repetition of steps 1) and 2) with N,N-dimethylformamide.

11. The method of claim 1, wherein the reagent is acetic anhydride.

12. The method of claim 1, wherein step (4) is carried out in the presence of a cutting agent comprising trifluoroacetic acid, triisopropylsilane, thioanisole, and water.
